(19) United States
(12) Patent Application Publication
(10) Pub. No.: US 2004/0176466 A1
(43) Pub. Date: Sep. 9, 2004

(54) Combination therapy for the treatment of neurological disorders

(76) Inventors: Albert John Allen, Indianapolis, IN (US); David Michelson, Carmel, IN (US); John-Michael Sauer, Indianapolis, IN (US)

Correspondence Address: Charles E Cohen, Eli Lilly & Company, Patent Division, PO Box 6288, Indianapolis, IN, 46206-6288, US

(21) Appl. No.: 10/484,646

(22) PCT Filed: Jul. 26, 2002
(86) PCT No.: PCT/US02/21294

(30) Foreign Application Priority Data
Aug. 8, 2001 (US) 60310981

(51) Int. Cl. A61K 31/445; A61K 31/137

(52) U.S. Cl. 514/649; 514/277

(57) ABSTRACT

The invention provides improved formulations and methods for the treatment of neurological disorders.

Atomoxetine, (R)-(−)-N-methyl-3-(2-methylphenoxy)-3-phenylpropylamine, is a selective inhibitor of norepinephrine uptake with little affinity for other uptake sites or neurotransmitter receptors (Gehlert, et al., Neuroscience Letters, 157, 203-206 (1993); Wong, et al., J. Pharmacol. Exp. Therap., 222, 61-65 (1982)). Atomoxetine has been investigated for the treatment of depression (Chouinard, et al., Psychopharmacology, 83, 126-128 (1984)), and has been reported to be efficacious for the treatment of attention deficit/hyperactivity disorder (ADHD) in adults (Spencer, et al., American Journal of Psychiatry, 155(5), 693-695 (1998)). Atomoxetine is currently being evaluated clinically for the treatment of ADHD.

Atomoxetine is primarily metabolized in humans by cytochrome P450 2D6 (CYP2D6). Cytochrome P450s generally comprise the major enzymes responsible for oxidative metabolism of drugs (Eichelbaum and Gross, Pharmacol. Ther., 46, 377 (1990)). The CYP2D6 enzyme specifically has a wide range of activity within human populations, with inter-individual rates of metabolism differing by more than 10,000 fold (McElroy, et al., AAPS Pharmsci. 2000, 2(4), Article 33 (http://www.pharmsci.org)). Most individuals are extensive metabolizers, able to metabolize CYP2D6 substrates extensively, whereas 7-10% of Caucasian individuals are poor metabolizers, producing no functional CYP2D6 enzyme. Poor metabolizers across all populations, including Asians and African Americans, comprise 2-10% (DeVane, The American Journal of Medicine, 97(Suppl. 6A), 6A-19S (1994)). A human pharmacokinetic study of atomoxetine revealed two distinct classes of kinetic disposition (Farid, et al., The Journal of Clinical Pharmacology, 25(4), 296-301 (1985)). In a majority of patients, atomoxetine exhibited a mean half-life of 4.5±1.1 hours, whereas atomoxetine had a half-life of 17.1 and 21 hours in two patients.

Inter-individual variability in drug metabolism poses a challenge in predicting dosing, safety, and efficacy of a drug. Pharmacokinetic factors, as well as substantial intersubject pharmacodynamic variability, have been proposed as a factor in cases of therapeutic failure of methylphenidate (DeVane, et al., Journal of Clinical Psychopharmacology, 20(3), 347 (2000)). In a recent study, atomoxetine was demonstrated to be robustly better than placebo in the treatment of ADHD, regardless of whether the patients' CYP2D6 status was as an extensive or poor metabolizer. Surprisingly, poor metabolizer ADHD patients demonstrated a greater response to atomoxetine treatment, most improving to the point of being clinically asymptomatic.

The present invention provides methods and formulations for addressing inter-individual variability in the CYP2D6-mediated metabolism of atomoxetine.

The present invention provides a method for decreasing inter-individual variability due to CYP2D6-mediated metabolism in the inhibition of norepinephrine uptake, comprising administering to a human that is a CYP2D6 extensive-metabolizer in need of inhibition of norepinephrine uptake an effective amount of atomoxetine in combination with an inhibitor of CYP2D6.

The present invention also provides a method for decreasing inter-individual variability due to CYP2D6-mediated metabolism in the inhibition of norepinephrine uptake, comprising the steps of:

a) determining the CYP2D6 status of a human in need of inhibition of norepinephrine uptake; and

b) administering to a human that is a CYP2D6 extensive-metabolizer in need of inhibition of norepinephrine uptake an effective amount of atomoxetine in combination with an inhibitor of CYP2D6.

The present invention further provides an improved method for the inhibition of norepinephrine uptake in a human by the administration of an effective amount of atomoxetine to a human in need of said inhibition, wherein the improvement comprises the co-administration of an inhibitor of CYP2D6.

The present invention also provides a method for the treatment of treatment-resistant attention deficit/hyper-activity disorder, comprising administering to a patient who has previously not responded to attention deficit/hyper-activity disorder treatment, an effective amount of atomoxetine in combination with an inhibitor of CYP2D6.

A further embodiment of the present invention is a method for increasing the mean plasma half-life of atomoxetine in a human, comprising administering to a human in need of inhibition of norepinephrine uptake an effective amount of atomoxetine in combination with an inhibitor of CYP2D6.

The present invention further provides a method for increasing the maximum steady state plasma concentration of atomoxetine in a human, comprising administering to a human in need of inhibition of norepinephrine uptake an effective amount of atomoxetine in combination with an inhibitor of CYP2D6.

The present invention also provides a pharmaceutical formulation comprising atomoxetine and an inhibitor of CYP2D6 in combination with a pharmaceutically acceptable excipient.

This invention also provides the use of atomoxetine in combination with an inhibitor of CYP2D6 for the manufacture of a medicament useful for the inhibition of norepinephrine uptake in a human. Additionally, this invention provides a pharmaceutical formulation adapted for the inhibition of norepinephrine uptake in a human containing atomoxetine in combination with an inhibitor of CYP2D6.

The present invention requires the co-administration of atomoxetine with an inhibitor of CYP2D6. Atomoxetine, which is also known in the art as tomoxetine, is (R)-(−)-N-methyl-3-(2-methylphenoxy)-3-phenylpropylamine, and is usually administered as the hydrochloride salt. Atomoxetine was first disclosed in U.S. Pat. No. 4,314,081. A convenient synthesis of atomoxetine is described in WO 00/61540. The word “atomoxetine” will be used here to refer to any acid addition salt or the free base of the molecule.

Many compounds are known to the skilled artisan to possess CYP2D6 inhibitory activity, and no doubt many more will be identified in the future (Pollock, Harvard Rev. Psychiatry, 2, 206 (1994); and Otton, et al., Clin. Pharmacol. Ther., 53, 401 (1993)). Methods for determining the ability of a compound to inhibit CYP2D6 are standard metabolic assays well known to the skilled artisan (See: Stephens and Wrighton, Journal of Pharmacology and Experimental Therapeutics, 266(2), 964-971 (1993); Otten, et al., Clinical Pharmacology and Therapeutics, 53(4), 401-409 (1993); and Crewe, et al., British Journal of Clinical Pharmacology, 34, 262-265 (1992)). An inhibitor of CYP2D6 is taken to be a compound that inhibits CYP2D6 activity by at least 50% at a pharmacologically acceptable dose. A pharmacologically acceptable dose is a dose that inhibits CYP2D6 activity without causing unacceptable side effects. It is preferred that the CYP2D6 inhibitor inhibits CYP2D6 activity by at least 75%. It is more preferred that the CYP2D6 inhibitor inhibits CYP2D6 activity by at least 80%. It is most preferred that the CYP2D6 inhibitor inhibits CYP2D6 activity to the level of a poor metabolizer.

The following compounds are examples of inhibitors of CYP2D6 useful for the methods and formulations of the present invention:

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine. In this document, the word “fluoxetine” will be used to mean any acid addition salt or the free base, and to include either the racemic mixture or either of the R and S enantiomers or any mixture thereof;

Norfluoxetine, 3-(p-trifluoromethylphenoxy)-3-phenylpropylamine, is a metabolite of fluoxetine and is a racemic mixture of its two enantiomers. U.S. Pat. No. 4,313,896 is an early reference to the compound. (S)-norfluoxetine is described in U.S. Pat. No. 5,250,571. (R)-norfluoxetine is described in U.S. Pat. No. 5,250,572. In this document, the word “norfluoxetine” will be used to mean any acid addition salt or the free base, and to include either the racemic mixture or either of the R and S enantiomers or any mixture thereof;

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985); and

Sertraline, (1S-cis)-4-(3,4-dichlorophenyl)-1,2,3,4-tetrahydro-N-methyl-1-naphthylamine hydrochloride, is a serotonin reuptake inhibitor that is marketed as an antidepressant. Sertraline is disclosed in U.S. Pat. No. 4,536,518.

All of the U.S. patents that have been mentioned above in connection with compounds used in the present invention are incorporated herein by reference.

It will be understood that while the use of a single inhibitor of CYP2D6 is preferred, combinations of two or more inhibitors of CYP2D6 may be used if necessary or desired. While all combinations of atomoxetine and inhibitors of CYP2D6 are useful and valuable, certain combinations are particularly valued and are preferred, as follows:

atomoxetine/fluoxetine

atomoxetine/fluoxetine hydrochloride

atomoxetine/(R)-fluoxetine

atomoxetine/(R)-fluoxetine hydrochloride

atomoxetine/(S)-fluoxetine

atomoxetine/(S)-fluoxetine hydrochloride

atomoxetine/norfluoxetine

atomoxetine/norfluoxetine hydrochloride

atomoxetine/(R)-norfluoxetine

atomoxetine/(R)-norfluoxetine hydrochloride

atomoxetine/(S)-norfluoxetine

atomoxetine/(S)-norfluoxetine hydrochloride

atomoxetine/paroxetine

atomoxetine/sertraline

In general, combinations and methods of treatment using fluoxetine or norfluoxetine as the CYP2D6 inhibitor are preferred. Especially preferred are combinations and methods of treatment using fluoxetine hydrochloride as the CYP2D6 inhibitor. In all instances, it is preferred that atomoxetine is atomoxetine hydrochloride.

In one embodiment of the present invention it is necessary to determine the CYP2D6 status of a human prior to the administration of atomoxetine in combination with an inhibitor of CYP2D6. As previously discussed, the CYP2D6 status is either that of an extensive-metabolizer or a poor-metabolizer. The determination of CYP2D6 status may be accomplished by methods well known to the skilled artisan. The determination of CYP2D6 status may be determined by either measuring the rate of metabolism of a CYP2D6 substrate (See: Stephens and Wrighton, Journal of Pharmacology and Experimental Therapeutics, 266(2), 964-971 (1993); Otten, et al., Clinical Pharmacology and Therapeutics, 53(4), 401-409 (1993); and Crewe, et al., British Journal of Clinical Pharmacology, 34, 262-265 (1992)), or by genotype and phenotype analysis (See: Jacqz, et al., Eur. J. Clin. Pharmacol., 35, 167 (1988); and Kupfer, et al., Lancet, 2, 517 (1984)).

Another embodiment of the present invention provides a method for increasing the mean plasma half-life of atomoxetine (T_{1/2}) in a human. The skilled artisan will appreciate that the T_{1/2} is the time required for the plasma concentration to be reduced by 50% (See: Goodman and Gilman, The Pharmacological Basis of Therapeutics, Ninth Edition, pages 21-22, McGraw-Hill, New York (1996)). Although any statistically significant increase in T_{1/2} is a useful result of the method of the present invention, it is preferred that the T_{1/2} is increased by at least two-fold by the method of the present invention relative to the administration of atomoxetine alone.

A further embodiment of the present invention provides a method for increasing the maximum steady state plasma concentration (C_{ss,max}) of atomoxetine in a human. The skilled artisan will appreciate that the C_{ss,max} is the maximum plasma concentration of atomoxetine achieved at steady state. Steady state is the point at which drug elimination equals the rate of drug availability (Goodman and Gilman, page 22). Although any statistically significant increase in C_{ss,max} is a useful result of the method of the present invention, it is preferred that the C_{ss,max} is increased by at least three-fold by the method of the present invention relative to the administration of atomoxetine alone.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

Many of the compounds used in this invention are amines, and accordingly react with any of a number of inorganic and organic acids to form pharmaceutically acceptable acid addition salts. Since some of the free amines of the compounds of this invention are typically oils at room temperature, it is preferable to convert the free amines to their pharmaceutically acceptable acid addition salts for ease of handling and administration, since the latter are routinely solid at room temperature. Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-1-sulfonate, naphthalene-2-sulfonate, mandelate and the like. Preferred pharmaceutically acceptable salts are those formed with hydrochloric acid.

The dose of drugs used in the present invention must, in the final analysis, be set by the physician in charge of the case based on knowledge of the drugs, the properties of the drugs in combination as determined in clinical trials, and the characteristics of the patient, including diseases other than that for which the physician is treating the patient. General outlines of the dosages, and some preferred dosages, can and will be provided here. Dosage guidelines for some of the drugs will first be given separately; in order to create a guideline for any desired combination, one would choose the guidelines for each of the component drugs.

Atomoxetine: from about 5 mg/day to about 200 mg/day; preferably in the range from about 60 to about 150 mg/day; more preferably from about 60 to about 130 mg/day; and still more preferably from about 60 to about 120 mg/day;

Fluoxetine: from about 1 to about 80 mg, once/day; preferred, from about 10 to about 40 mg once/day;

Norfluoxetine: from about 0.01-20 mg/kg once/day; preferred, from about 0.05-10 mg/kg once/day, most preferred, from about 0.1-5 mg/kg once/day;

Paroxetine: from about 20 to about 50 mg once/day; preferred, from about 20 to about 30 mg once/day.

Sertraline: from about 20 to about 500 mg once/day; preferred, from about 50 to about 200 mg once/day;

In more general terms, one would create a combination of the present invention by choosing a dosage of atomoxetine and CYP2D6 inhibitor component compounds according to the spirit of the above guideline.

The adjunctive therapy of the present invention is carried out by administering atomoxetine in combination with an inhibitor of CYP2D6 in any manner that provides effective levels of the compounds in the body at the same time. All of the compounds concerned are orally available and are normally administered orally, and so oral administration of the adjunctive combination is preferred. They may be administered together, in a single dosage form, or may be administered separately.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form that is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound that provides the desired dose to the patient in need of such treatment. The activity of the adjunctive combinations does not depend on the nature of the composition, so the compositions are chosen and formulated solely for convenience and economy. Any of the combinations may be formulated in any desired form of composition.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium, and calcium stearate, stearic acid and hydrogenated vegetable oils.

Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

The present invention provides the advantage of treatment of neurological disorders with atomoxetine without the inter-patient variability in metabolism typically observed with such treatment, conferring a marked and unexpected benefit on the patient.

The formulations and methods of the present invention are particularly suited for use in the treatment of attention deficit/hyperactivity disorder (ADHD), depression, anxiety disorders, obsessive compulsive disorder, urinary incontinence, enuresis, oppositional defiant disorder, and conduct disorder. Such disorders may often be resistant to treatment with atomoxetine alone. The titles given many of these conditions represent multiple disease states. The following list illustrates a number of these disease states, many of which are classified in the Diagnostic and Statistical Manual of Mental Disorders, 4th Edition, published by the American Psychiatric Association (DSM). The DSM code numbers for these disease states are supplied below, when available, for the convenience of the reader.

| | |
|---|---|
| ADHD, Combined Type | DSM 314.01 |
| ADHD, Predominantly Inattentive Type | DSM 314.00 |
| ADHD, Predominantly Hyperactive- | DSM 314.01 |
| Impulsive Type | |
| ADHD, Not Otherwise Specified | DSM 314.9 |
| Conduct Disorder, Child-Onset Type | DSM 312.81 |
| Conduct Disorder, Adolescent-Onset Type | DSM 312.82 |
| Conduct Disorder, Unspecified Onset | DSM 312.89 |
| Oppositional Defiant Disorder | DSM 313.81 |
| Major Depressive Episode, | DSM 296.2x |
| Single Episode | |
| Major Depressive Episode, Recurrent | DSM 296.3x |
| Dysthymic Disorder | DSM 300.4 |
| Panic Disorder Without Agoraphobia | DSM 300.01 |
| Panic Disorder With Agoraphobia | DSM 300.21 |
| Agoraphobia Without History of Panic | DSM 300.22 |
| Disorder | |
| Specific Phobia | DSM 300.29 |
| Social Phobia | DSM 300.23 |
| Obsessive-Compulsive Disorder | DSM 300.3 |
| Post-Traumatic Stress Disorder | DSM 309.81 |
| Acute Stress Disorder | DSM 308.3 |
| Generalized Anxiety Disorder | DSM 300.02 |
| Anxiety Disorder Due to a General Medical | DSM 293.84 |
| Condition | |
| Substance Induced Anxiety Disorder | |
| Alcohol | DSM 291.89 |
| Amphetamine (or Amphetamine-Like | DSM 292.89 |
| Substance) | |
| Caffeine | DSM 292.89 |
| Cannabis | DSM 292.89 |
| Cocaine | DSM 292.89 |
| Hallucinogen | DSM 292.89 |
| Inhalant | DSM 292.89 |
| Phencyclidine (or Phencyclidine-Like | DSM 292.89 |
| Substance) | |
| Sedative, Hypnotic, or Anxiolytic | DSM 292.89 |
| Other [Unknown] Substance | DSM 292.89 |
| Anxiety Disorder Not Otherwise | DSM 300.00 |
| Specified | |
| Separation Anxiety Disorder | DSM 309.21 |
| Sexual Adversion Disorder | DSM 302.79 |
| Enuresis | DSM 307.6 |

Urinary incontinence is generally defined as the involuntary loss of urine and is most common in children, women, the elderly, and neurological disease patients. Stress incontinence is the involuntary loss of urine through an intact urethra produced during times of increased abdominal pressure such as during physical activity and coughing. The loss of urine is not accompanied by premonitory sensations of the need to void and is not related to the fullness of the bladder. Urge incontinence is the involuntary loss of urine through an intact urethra due to an increased intrabladder pressure. In contrast to stress incontinence, urge incontinence is caused by an episodic bladder contraction (detrusor instability) that exceeds the outlet resistance pressure generated by the urethra, and is accompanied by a perception of urgency to void. Complex incontinence has the characteristics of both urge and stress incontinence.

The method of the present invention is effective in the treatment of patients who are children, adolescents or adults, and there is no significant difference in the symptoms or the details of the manner of treatment among patients of different ages. In general terms, however, for purposes of the present invention, a child is considered to be a patient below the age of puberty, an adolescent is considered to be a patient from the age of puberty up to about 18 years of age, and an adult is considered to be a patient of 18 years or older.

### EXAMPLE 1

### Combination of Atomoxetine and Paroxetine

Subjects

The study was conducted at the Lilly Laboratory for Clinical Research in Indianapolis, Ind. The protocol and informed consent documents were approved by the Institutional Review Board of Indiana University—Purdue University at Indianapolis. The study was conducted in accordance with the Declaration of Helsinki. All participants provided informed written consent before enrollment into the study. All volunteers were considered to be healthy on the basis of medical history, electrocardiographic findings, and routine clinical laboratory tests. Volunteers with clinically abnormal results were excluded from the study.

Only CYP2D6 extensive metabolizers, as determined by genotyping and phenotyping analyses, were entered in this study. CYP2D6 genotype was performed by PPGx (Morrisville, N.C.). DNA from whole blood samples were isolated and purified and analyzed for CYP2D6 genotype using a validated PCR (polynucleotide chain reaction) method. CYP2D6 genotype was evaluated by testing the *3, *4, *5, *6, *7, and *8 poor metabolizer (PM) alleles. If patients were homozygous for any combination of these alleles, a PM genotype was assigned; otherwise, an extensive metabolizer genotype (EM) was assigned. CYP2D6 phenotype was performed using the urine ratio of dextromethorphan/dextrorphan following an oral dose of dextromethorphan. Volunteers with a ratio greater than 0.3 were assigned a PM phenotype, and those with a ratio less than 0.3 were assigned an EM phenotype.

Twenty-two subjects were entered into the study, and 14 subjects completed both treatment periods. There were 17 males and 5 females, ranging from 20 to 49 years of age with a mean age of 38 years. The mean BMI for women was 23.8 kg/m² and for men was 24.4 kg/m². Seven subjects were discontinued from the study by the physician due either to noncompliance or to the finding of a positive urine drug test. One participant withdrew for personal reasons.

Study Design

This was a single-blind, sequential study composed of two periods. In period one, volunteers received oral doses of 20-mg atomoxetine every twelve hours for nine doses. In period two, 20-mg paroxetine (Paxil, SmithKline Beecham Pharmaceuticals, Crawley, UK) was administered once daily with oral doses of placebo every twelve hours for days 1 through 11. Beginning the morning of day 12 and continuing through day 16, once-daily doses of paroxetine were coadministered with 20-mg atomoxetine every 12 hours. On day 17, the final oral doses of atomoxetine and paroxetine were coadministered in the morning. Doses were administered with 240 mL of water. Subjects were fasted overnight prior to administration of morning doses of atomoxetine or placebo and paroxetine, and breakfast was served no earlier than 60 minutes following administration. Subjects were fasted at least two hours (except for liquids) prior to administration of evening doses of atomoxetine or placebo, and evening meals were served no earlier than 60 minutes following administration.

Sample Collection

Period 1: Multiple Dose Atomoxetine. A trough plasma sample was obtained immediately prior to the 7^{th}, 8^{th}, and 9^{th} atomoxetine doses. Additional plasma samples were obtained after the 9^{th} dose of atomoxetine at 0.5, 1, 1.5, 2, 4, 6, 12, 18, and 24 hours postdose.

Period 2: Multiple Dose Paroxetine and Multiple Dose Atomoxetine.

A trough plasma sample was taken immediately prior to the 9^{th}, 10^{th}, and 11^{th} paroxetine doses. Additional plasma samples were obtained after the 11^{th} paroxetine dose at 0.5, 1, 1.5, 2, 4, 6, 12, 18, and 24 hours postdose. On Study Day 12 after the first dose of atomoxetine with paroxetine, plasma samples were obtained to evaluate atomoxetine pharmacokinetics at 1, 2, 4, 6, and 12 hours postdose. A trough plasma sample was taken immediately prior to the 15^{th}, 16^{th}, and 17^{th} paroxetine doses, and a trough plasma sample was taken immediately prior to the 9^{th}, 10^{th}, and 11^{th} atomoxetine doses. Additional plasma samples were obtained to evaluate both atomoxetine and paroxetine pharmacokinetic parameters after reaching steady state for the combination on Day 17 (17^{th} paroxetine dose and 11^{th} atomoxetine dose) at 0.5, 1, 1.5, 2, 4, 6, 8, 12, 18, 24, 36, 48, 72, 96, and 120 hours postdose.

Analytical Methods

Plasma samples were analyzed for atomoxetine, N-desmethylatomoxetine, and 4-hydroxyatomoxetine concentrations using a validated liquid chromatography/atmospheric pressure chemical ionization/mass spectrometry/mass spectrometry (LC/APCI/MS/MS) method over the concentration ranges 1 to 800 ng/mL for N-desmethylatomoxetine and 4-hydroxyatomoxetine and 2.5 to 2000 ng/mL for atomoxetine. If required, additional analyses were conducted using a lower range validated LC/APCI/MS/MS method over the concentration ranges 1 to 100 ng/mL for N-desmethylatomoxetine and 4-hydroxyatomoxetine and 0.25 to 25 ng/mL for atomoxetine (Taylor Technology, Inc, Princeton, N.J.).

Plasma samples were analyzed for paroxetine using a validated gas chromatograph/nitrogen phosphorus detector (GC/NPD) method over the concentration range 0.25 to 50 ng/mL (PPD Development, Richmond, Va.).

Pharmacokinetic Analysis

Pharmacokinetic parameter estimates were calculated with noncompartmental analysis by using WinNonlin Professional Version 2.1 (Pharsight Corp, Mountain View, Calif.). The steady state maximum plasma concentration (C_{ss,max}), and the corresponding time of the maximum concentration (Tₘₐₓ) were observed values. The elimination rate constant (λ_{z}) was determined as the slope of the linear regression for the terminal log-linear portion of the concentration-time curve. Terminal half-life (t_{1/2}) was calculated as ln(2)/λ_{z}. The area under the plasma concentration time curve (AUC_{0-τ}) over the dosing interval was estimated by the linear trapezoidal method. The dosing intervals (τ) for atomoxetine and paroxetine were 12 and 24 hours, respectively. Apparent clearance (CLₛₛ/F) and apparent volume of distribution (V_{z}/F) were calculated as Dose/AUC_{0-τ} and as (CLₛₛ/F)/λ_{z}, respectively.

Statistical Analysis

For atomoxetine and N-desmethylatomoxetine, the following parameters were evaluated for treatment differences: C_{ss,max}, AUC_{0-τ}, t_{1/2}, and Tₘₐₓ. For paroxetine, the following parameters were evaluated for treatment differences: C_{ss,max}, AUC_{0-τ}, and Tₘₐₓ. Except for Tₘₐₓ, all parameters were log transformed, and a mixed-effect analysis of variance was performed with subject as a random effect. Geometric means, ratio of geometric means, 90% confidence intervals of the ratios, and p-values for the hypothesis of no treatment differences were calculated. For Tₘₐₓ, a Wilcoxon sign-rank test was performed. Data from all subjects who received study drug are included in the pharmacokinetic and statistical analyses, except for the Tₘₐₓ analyses that excluded subjects with measurements for only one treatment. Statistical analyses were performed with SAS Version 6.12 (SAS Institute, Cary, N.C.).

Pharmacokinetics of Atomoxetine

Steady state atomoxetine plasma concentrations were higher after coadministration with paroxetine compared to atomoxetine administration alone. On the basis of visual examination of trough plasma atomoxetine concentrations, steady state was achieved in all subjects when the pharmacokinetic profile was obtained. Steady state trough atomoxetine concentrations ranged between 16.0 to 22.0 ng/mL in the absence of paroxetine, and 325 to 359 ng/mL in the presence of paroxetine. The steady state pharmacokinetic parameters of atomoxetine are presented in Table I. The coadministration with paroxetine to steady state led to a significant increase in the C_{ss,max} and AUC_{0-τ} values of atomoxetine by approximately 3.5- and 6.5-fold, respectively. The t_{1/2} for atomoxetine increased approximately 2.5-fold from 3.92 hours to 10.02 hours after concomitant paroxetine administration. The coadministration of paroxetine had a statistically significant shift in the Tₘₐₓ values for atomoxetine (p=0.0078). However, the median of the paired difference was 0.5 hours, and therefore considered clinically insignificant.

Administration of a therapeutic dose of paroxetine (20 mg once a day) for 17 days resulted in steady state plasma concentrations in the same range as its inhibitory constant for CYP2D6 (0.15 μM) as determined in vitro. Consequently, coadministration of paroxetine and atomoxetine led to an increase in the plasma concentrations of atomoxetine. Paroxetine increased mean steady state C_{ss,max} and AUC_{0-τ} values of atomoxetine by about 3.5- and 6.5-fold, respectively. Thus, dosing of paroxetine and atomoxetine to steady state resulted in atomoxetine pharmacokinetics similar to that of patients deficient in CYP2D6 activity.

TABLE I
Arithmetic mean (% CV) steady-state pharmacokinetic
parameters of atomoxetine in extensive metabolizers after
atomoxetine dosing alone and after coadministration of
atomoxetine with paroxetine

| | | Atomoxetine with |
|---|---|---|
| | Atomoxetine Alone | Paroxetine |
| | (Period 1) | (Period 2) |
| Atomoxetine | n = 21 | n = 14 |
| C_{ss, max} (ng/mL) | 184 (36) | 690 (37) |
| Tₘₐₓ^{a} (hr) | 1.00 (0.50-2.00) | 1.50 (0.50-4.00) |
| AUC_{0-τ} (μg · hr/mL) | 0.846 (45) | 5.97 (42) |
| T_{1/2}^{b} (hr) | 4.03 (2.87-7.20) | 11.0 (4.87-19.6) |
| CLₛₛ/F (L/hr/kg) | 0.395 (55) | 0.0599 (81) |
| V_{z}/F (L/kg) | 2.20 (50) | 0.803 (44) |

^{a}Median (range)
^{b}Mean (range)

We claim:

1. A method for decreasing inter-individual variability due to CYP2D6-mediated metabolism in the inhibition of norepinephrine uptake, comprising administering to a human that is a CYP2D6 extensive-metabolizer in need of norepinephrine uptake inhibition an effective amount of atomoxetine in combination with an inhibitor of CYP2D6.

2. A method for decreasing inter-individual variability due to CYP2D6-mediated metabolism in the inhibition of norepinephrine uptake, comprising the steps of:
a) determining the CYP2D6 status of a human in need of inhibition of norepinephrine uptake; and
b) administering to a human that is a CYP2D6 extensive-metabolizer in need of norepinephrine uptake inhibition an effective amount of atomoxetine in combination with an inhibitor of CYP2D6.

3. An improved method for the inhibition of norepinephrine uptake in a human by the administration of an effective amount of atomoxetine to a human in need of said inhibition, wherein the improvement comprises the co-administration of an inhibitor of CYP2D6.

4. A method for the treatment of treatment-resistant attention-deficit/hyperactivity disorder, comprising administering to a patient who has previously not responded to attention-deficit/hyperactivity disorder treatment, an effective amount of atomoxetine in combination with an inhibitor of CYP2D6.

5. A method for increasing the mean plasma half-life of atomoxetine in a human, comprising administering to a human in need of inhibition of norepinephrine uptake an effective amount of atomoxetine in combination with an inhibitor of CYP2D6.

6. A method for increasing the maximum steady state plasma concentration of atomoxetine in a human, comprising administering to a human in need of inhibition of norepinephrine uptake an effective amount of atomoxetine in combination with an inhibitor of CYP2D6.

7. A method of any of claims 1-6, where the inhibitor of CYP2D6 is selected from the group consisting of fluoxetine, norfluoxetine, paroxetine, and sertraline.

8. A method of claim 7, where the inhibitor of CYP2D6 is fluoxetine hydrochloride.

9. A method of claim 8, where atomoxetine is atomoxetine hydrochloride.

10. A pharmaceutical formulation comprising atomoxetine and an inhibitor of CYP2D6 in combination with a pharmaceutically acceptable excipient.

11. A formulation of claim 10, where the inhibitor of CYP2D6 is selected from the group consisting of fluoxetine, norfluoxetine, paroxetine, and sertraline.

12. A formulation of claim 11, where the inhibitor of CYP2D6 is fluoxetine hydrochloride.

13. A formulation of claim 12, where atomoxetine is atomoxetine hydrochloride.
